(19) United States
(12) Patent Application Publication
(10) Pub. No.: US 2013/0071872 A1
(43) Pub. Date: Mar. 21, 2013

(54) DEVICE AND METHOD FOR SINGLE-USED MATRIX SAMPLING

(71) Applicants: Lewis HO, Lawrenceville, GA (US); Karen Chen, Taichung (TW); King-Ming Chang, Taichung (TW)

(72) Inventors: Lewis HO, Lawrenceville, GA (US); Karen Chen, Taichung (TW); King-Ming Chang, Taichung (TW)

(21) Appl. No.: 13/617,414

(22) Filed: Sep. 14, 2012

Related U.S. Application Data
(60) Provisional application No. 61/627,062, filed on Sep. 15, 2011.

(51) Int. Cl. G01N 1/14; C12Q 1/02

(52) U.S. Cl. 435/29; 422/547

(57) ABSTRACT

A single-used matrix sampling device for the single-used, disposable or autoclavble packed-bed bioreactor is provided herein. The present invention includes a tube means and a matrix collecting means. The matrix collecting means configured for collecting the cell culture matrix includes a receptacle and a pulling means connected to the receptacle or the matrix collecting means could be scoop-like in configuration; the tube means is configured for preventing the matrix collecting means from being contaminated while mechanically or pneumatically sampling; and the tube means could be sealed to enclose the sampled matrix collecting means after sampling. A single-used matrix sampling method for a single-used, disposable or autoclavble packed-bed bioreactor is also provided. The present invention could be utilized to aseptically collect the cell culture matrix from the packed-bed bioreactor without requiring to face the risks of contamination.

### RELATED APPLICATION/PATENTS & INCORPORATION BY REFERENCE

This application claims priority to provisional application Ser. No. 61/627,062 filed Sep. 15, 2011 entitled “Matrix sampling device” incorporated herein by reference.

### BACKGROUND OF THE INVENTION

1. Field of the Invention

The present invention relates generally to a single-used matrix sampling technique designed for cell culture product, and, more particularly to a single-used matrix sampling device and method for sampling the cell culture matrixes aseptically from the single-used, disposable or autoclavble packed-bed bioreactor.

2. Description of the Prior Art

Single-used or disposable products have becoming popular in pharmaceutical industries due to the elimination of cleaning validation, reduce cost and increase turnover rate for production. Bioreactors, especially those for animal or mammalian cell culture, are also toward the same trend that provides the similar advantages as other disposable products. There are many types of cell culture bioreactors, such as stir tank bioreactors, or packed-bed bioreactors. From all types of bioreactors, a packed-bed bioreactor is more difficult to develop as a disposable product than the others due to regulation issues. Traditional packed-bed bioreactor is unable to sample the cell culture matrixes from the bioreactor system and it limits the application in industrial scale production purpose due to the requirement of analyzing cell characteristics during the cell culture process especially for the producing of human vaccines or therapeutic biopharmaceuticals. Therefore, a design of single-used matrix sampling for packed-bed bioreactor is necessary for the system be applied in industrial manufacture purpose.

### SUMMARY OF THE INVENTION

The present invention is directed to a single-used matrix sampling device and method which uses a tube means and a matrix collecting means for sampling the cell culture matrixes aseptically from the single-used, disposable or autoclavble packed-bed bioreactor in order to do cell analysis including cell count, viability, contamination, cell characterization, . . . etc. without requiring to face the threaten of contamination.

The cell culture single-used matrix sampling device contains a rigid and/or flexible tube and a matrix collecting device. The matrix collecting device can be mechanical or pneumatically, can be extended into the packed-bed to collect the matrix sample. After the samples are mechanically collected, the device along with the collected sample can be enclosed inside the flexible tube which can then be sealed to enclose the matrix collecting device with sample. Or the sample can be pneumatically collected into a collecting bottle through the flexible tube which can be also sealed to enclose the sample. The sample collected can be removed aseptically for further analysis.

According to one embodiment, a single-used matrix sampling device for a packed-bed bioreactor which comprises a closed container to receive a cell culture matrix, the single-used matrix sampling device including: a matrix collecting means and a tube means. The matrix collecting means is configured for sampling the cell culture matrix, the matrix collecting means includes a receptacle and a pulling means configured to the receptacle at one end thereof for moving the receptacle outside the container. The tube means is configured for preventing the matrix collecting means from being contaminated while sampling, and the tube means connects to the container at one end with a connector, wherein the tube means is sealed to enclose the receptacle after the receptacle is pulled outside the container through the connector to the tube means.

According to another embodiment, a single-used matrix sampling device for a packed-bed bioreactor which comprises a container which is closed to receive a cell culture matrix, the single-used matrix sampling device comprising a matrix collecting means and a tube means. The matrix collecting means is configure for sampling the cell culture matrix, wherein the matrix collecting means comprises a matrix container equipped with a vacuum means and a sampling rod; and the sampling rod is extended into the container surrounded with the cell culture matrix through an opening on the container to guide the cell culture matrix sucked into the matrix container. The tube means is configured for preventing the matrix collecting means from being contaminated while sampling, wherein the tube means is connected between the sampling rod and the matrix container; and the tube means is sealed to enclose the matrix container after the cell culture matrix is sucked out the container through the connector to the matrix container by the vacuum means.

According to another embodiment, a single-used matrix sampling method comprising the following step. First, a single-used matrix sampling device is provided for a packed-bed bioreactor which comprises a container which is closed to receive a cell culture matrix, the single-used matrix sampling device comprising: a matrix collecting means for sampling the cell culture matrix, wherein the matrix collecting means comprises a matrix container equipped with a vacuum means and a sampling rod, wherein the sampling rod is extended into the container surrounded with the cell culture matrix through an opening on the container to guide the cell culture matrix sucked into the matrix container; and a tube means for preventing the matrix collecting means from being contaminated while sampling, wherein the tube means is connected between the sampling rod and the matrix container; and the tube means is sealed to enclose the matrix container after the cell culture matrix is sucked out the container through the connector to the matrix container by the vacuum means. Secondly, the matrix collecting means is put into the container to submerge the sampling rod in the cell culture matrix. Then, the cell culture matrix is vacuumed out from the container through the connector to the matrix container by the vacuum means. The tube means is sealed to enclose the matrix container to form an enclosed matrix sample by a heat sealing method or a compressing method, and the sealed zone of the tube means is cut off to separate the sealed tube means from the packed-bed bioreactor. Finally, the vacuum means is removed and the enclosed matrix sample is taken out from the sealed tube means for further analysis. In one embodiment, the selection of the seal device for the tube means is as follows. If the packed-bed bioreactor is a single-used packed-bed bioreactor, the tube means can be a thermal settable tube, such as a C-Flex tube, which can be sealed by a heat sealer; and if the packed-bed bioreactor is an autoclavble packed-bed bioreactor, the tube means can be a heat-resisting thermal settable tube, such as a platinum-cured silicon tube, which can be sealed via compressing the tube means by a clamp.

According to yet another embodiment, a single-used matrix sampling method comprising the following step. First, a single-used matrix sampling device is provided for a packed-bed bioreactor which comprises a container which is closed to receive a cell culture matrix, the single-used matrix sampling device comprising: a matrix collecting means for sampling the cell culture matrix, wherein the matrix collecting means comprises a matrix container equipped with a vacuum means and a sampling rod, wherein the sampling rod is extended into the container surrounded with the cell culture matrix through an opening on the container to guide the cell culture matrix sucked into the matrix container; and a tube means for preventing the matrix collecting means from being contaminated while sampling, wherein the tube means is connected between the sampling rod and the matrix container; and the tube means is sealed to enclose the matrix container after the cell culture matrix is sucked out the container through the connector to the matrix container by the vacuum means. Secondly, the matrix collecting means is put into the container to submerge the sampling rod in the cell culture matrix. Then, the cell culture matrix is vacuumed out from the container through the connector to the matrix container by the vacuum means. The tube means is sealed to enclose the matrix container to form an enclosed matrix sample by a heat sealing method or a compressing method, and the sealed zone of the tube means is cut off to separate the sealed tube means from the packed-bed bioreactor. Finally, the vacuum means is removed and the enclosed matrix sample is taken out from the sealed tube means for further analysis. In one embodiment, the selection of the seal device for the tube means is as follows. If the packed-bed bioreactor is a single-used packed-bed bioreactor, the tube means can be a thermal settable tube, such as a C-Flex tube, which can be sealed by a heat sealer; and if the packed-bed bioreactor is an autoclavble packed-bed bioreactor, the tube means can be a heat-resisting thermal settable tube, such as a platinum-cured silicon tube, which can be sealed via compressing the tube means by a clamp.

The objective, technologies, features and advantages of the present invention will become more apparent from the following description in conjunction with the accompanying drawings, wherein certain embodiments of the present invention are set forth by way of illustration and examples.

### BRIEF DESCRIPTION OF THE DRAWINGS

The following description, given by way of example, is not intended to limit the invention to any specific embodiments described. The description may be understood in conjunction with the accompanying Figures, incorporated herein by reference.

FIGS. 1 to 5 are side views illustrating the single-used matrix sampling device of one embodiment of the present invention;

FIGS. 6 to 10 are side views illustrating the single-used matrix sampling device according to another embodiment of the present invention;

FIGS. 11 to 15 are side views illustrating the single-used matrix sampling device according to another embodiment of the present invention;

FIGS. 16 to 18 are side views illustrating the single-used matrix sampling device according to another embodiment of the present invention;

FIGS. 19 to 22 are side views illustrating the single-used matrix sampling device according to another embodiment of the present invention;

FIGS. 23 to 27 are side views illustrating the single-used matrix sampling device according to another embodiment of the present invention;

FIG. 28 is an enlarged schematic diagram illustrating the matrix collecting means for the embodiment of the present invention;

FIGS. 29A, 29B and 29C are schematic diagrams respectively illustrating the side view, the 3D perspective view and the 3D view of the matrix collecting device for horizontal direction for another embodiment of the present invention; and

FIGS. 30A, 30B and 30C are schematic diagrams respectively illustrating the front view, the 3D perspective view and the 3D view of the matrix collecting device for vertical direction for another embodiment of the present invention.

### DESCRIPTION OF THE PREFERRED EMBODIMENT

The following detailed description, given by way of example, is not intended to limit the invention to any specific embodiments described. The detailed description may be understood in conjunction with the accompanying figures, incorporated herein by reference. Without wishing to unnecessarily limit the foregoing, the following shall discuss the invention with respect to certain preferred embodiments.

The invention describes a sampling device for aseptically collecting cell culture matrixes from a packed-bed bioreactor. The sampling device is constructed majorly by two parts: one is a flexible tube; the other is a matrix collecting device. The flexible tube could be a thermal settable tube such as a C-Flex tube or a heat-resisting thermal settable tube such as a silicon tube. It could also be a tube made by plastic film, or could be a tube with bellows so that the length of the tube could be shorten or extended. The matrix collecting device can be a forceps, a holder with one or more openings, or a scoop to collect the matrixes inside the packed-bed bioreactor, or a basket containing pre-embedded cell culture matrixes. The matrix collecting device could also be a rigid tube connected to a matrix container with air filter where the cell culture matrixes could be sucked out by vacuum through the air filter with a vacuum machine. The matrix collecting device could be placed inside the packed-bed bioreactor together with the cell culture matrixes and connected by pulling a string or a rigid rod and draw the matrix collecting device into the flexible tube where the tube is placed outside the packed-bed bioreactor. The matrix collecting device could be placed outside of the packed-bed bioreactor and can reach into the packed-bed bioreactor through a flexible tube to collect the matrix samples. The flexible tube is placed outside the packed-bed bioreactor in order to protect the matrix collecting device and to enclose the matrix collecting device after sampling. The flexible tube can also be placed inside the packed-bed bioreactor where the matrix collecting device is connected at the end of the flexible tube, and it could be pulled out of the packed-bed bioreactor and the tube turn inside out to enclose the matrix collecting device. After the matrix collecting device is covered by the flexible tube by either pulling the flexible tube to make it inside out, or to pull the matrix collecting device into a flexible tube by a string or a rigid rod, the matrix collecting device could be enclosed by sealing the flexible tube above and under the matrix collecting device. Then, after cutting off the sealing zone, the matrix collecting device containing collected matrix sample could be removed from the packed-bed bioreactor and protected by the sealed flexible tube. Therefore, a sampling of the cell culture matrix without posing the risk of contamination could be achieved.

The first embodiment of the present invention of the single-used matrix sampling device contains a matrix collecting device that is a basket (matrix sample basket) pre-embedded with cell culture matrixes that is placed in the packed-bed bioreactor together with other cell culture matrixes but separated by a rigid tube with openings that allows cells and nutrients to flow freely among the matrixes and the tube. The matrix sample basket containing cell culture matrixes is connected with a string. The string is extended through a rigid tube with openings inside the packed-bed bioreactor container and a flexible tube outside the packed-bed bioreactor container and fixed with a closure on top of the flexible tube. The closure is made with robber with resilient opening such as a needleless injection port. The opening in the closure is kept seal tight to prevent contaminants to pass. During sampling, the matrix sample basket is pulled up by the string and move the basket from the rigid tube with openings into the flexible tube above the packed-bed bioreactor container. The flexible tube from above and under the matrix sample basket is sealed to enclose the matrix basket by a sealing device. Then the enclosed matrix sample basket is removed for analysis by cutting off from the sealed tube. In one embodiment, the selection of the sealing device for the tube means is as follows. If the packed-bed bioreactor is a single-used packed-bed bioreactor, the tube means can be a thermal settable tube, such as a C-Flex tube, which can be sealed by a heat sealer; and if the packed-bed bioreactor is an autoclavble packed-bed bioreactor, the tube means can be a heat-resisting thermal settable tube, such as a platinum-cured silicon tube or another silicon tube, which can be sealed via compressing the tube means by a clamp. Besides, in another embodiment, a metallic collar is molded to the silicon tube, and a cutter compresses the collar and the silicon tube to create the sterile seal. To collect matrix sample randomly inside the packed-bed bioreactor, multiple single-used matrix sampling devices could be applied in different vertical and horizontal position so that cell culture matrixes in different positions could be collected for analysis purpose. Additionally, the matrix sample basket could be replaced to a holder with single or multi-openings that could collect the matrixes in the packed-bed bioreactor from different positions during the pulling motion. The rigid tube with openings for guiding the basket or holder can be replaced to a guide with a shape of funnel mounted under the flexible tube.

The second embodiment of the present invention of the single-used matrix sampling device contains a matrix collecting device that is a basket containing pre-embedded matrixes that connected together with a flexible tube make by a thin plastic film. The single-used matrix sampling device can be mounted on top or side of the packed-bed bioreactor. The flexible tube can be pulled out from the inner of the packed-bed bioreactor container to outer of the container with a string or a rod that seal at the end of the flexible tube that connected with the matrixes for sampling. After pulling the flexible tube from inner container to outer container, the flexible tube becomes inside out and the matrixes will be covered by the flexible tube. A tube sealer or other sealing device could be applied to seal the tube and form a close chamber containing the sampled matrixes and separated the sample from the packed-bed bioreactor container. The matrix sample basket could be replaced to a holder with single or multi-openings that could collect the matrixes from different positions in the packed-bed bioreactor during the pulling motion.

The third embodiment of the invention of the single-used matrix sampling device contains a rigid tube with opening that connected with a holder. The rigid tube is extended into the packed-bed bioreactor surrounded with cell culture matrix. Outside of the rigid tube is covered with a flexible tube with bellows. The rigid tube can move to and fro in the packed-bed bioreactor to collect the matrixes from different regions by pushing and pulling the bellows. The container connected with the rigid tube has an air filter. A vacuum machine can connect to the air filter on the container and generate a vacuum in the container to produce a suction force to sucking the matrix out from the packed-bed bioreactor through the rigid tube with opening. After collecting the cell culture matrixes, a tube sealer or other sealing device could be applied to seal the flexible tube and form a close chamber containing the sampled matrixes and separated the sample from the packed-bed bioreactor container.

The fourth embodiment of the invention of the single-used matrix sampling device contains a flexible tube with bellows, a scoop with rod or rigid but flexible tube is inside the flexible tube. The flexible tube is connected with a sterile connector such as ReadyMat or Kleenpak sterile connectors. An opening on the packed-bed bioreactor is also connected with a sterile connector such as ReadyMat or Kleenpak sterile connectors. After connect the opening on the packed-bed bioreactor with the single-used matrix sampling device, the scoop could be extended into the packed-bed bioreactor to collect the cell culture matrixes and pulled back into the flexible tube that will surround the scoop containing collected cell culture matrixes. After collecting the cell culture matrixes, a tube sealer or other sealing device could be applied to seal the flexible tube and form a close chamber containing the sampled matrixes and separated the sample from the packed-bed bioreactor container.

Reference is now made to the figures by way of examples and they are by no way limiting the scope of the present invention.

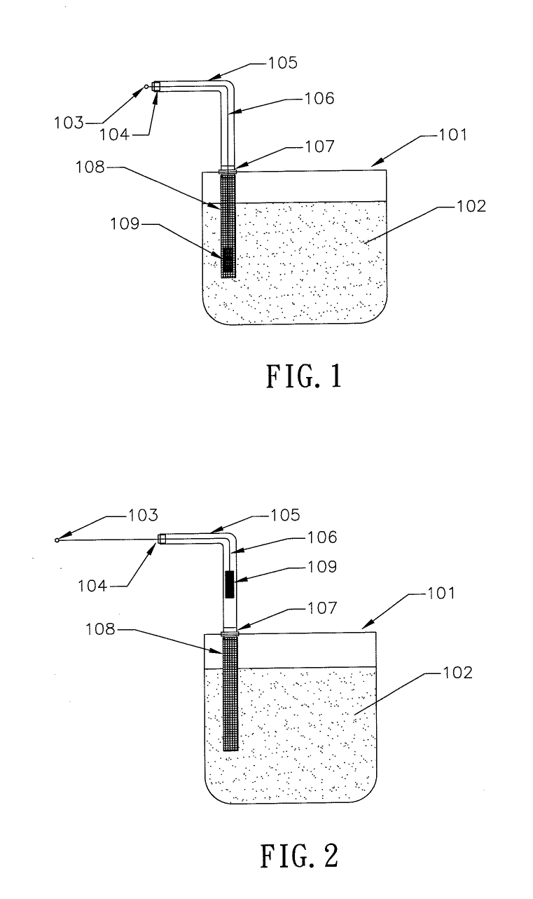

Referring to FIG. 1, the first embodiment of the present invention where the packed-bed bioreactor container 101 with packed cell culture matrixes 102 inside the container 101. The single-used matrix sampling device is constructed by a knob 103 for pulling the matrix sample basket 109. The knob 103 is connected with a string 106 through a closure 104. The closure 104 is preferably a robber stopper with a resilient opening that the string 106 is passed. A flexible tube 105 is mounted outside the container 101. The flexible tube 105 is preferably made by C-flex tube which can be sealed by heat. Or other materials that could be sealed by a sealer or other sealing means. Inside the packed-bed bioreactor container 101, there is a rigid tube 108 with openings that allows the cells and nutrient to flow freely inside and outside the rigid tube 108. The matrix sample basket 109 contains pre-embedded matrixes for sampling purpose is placed inside the rigid tube 108. The rigid tube 108 and the flexible tube 105 is connected with a connector 107 with two end openings, usually a panel mount connector.

Referring to FIG. 2, when the sampling of cell culture matrix is required, the string 106 is pulled by pulling the knob 103 through the closure 104 and the matrix sample basket 109 containing the cell culture matrixes is pulled into the flexible tube 105 outside the packed-bed bioreactor container 101 through the guiding of the rigid tube 108 inside the container 101. In another embodiment, not shown in the figure, the flexible tube is made by plastic film, wherein one end of the flexible tube is connected to the connector and the other end of the flexible tube is closed so that the string can be pulled inside the flexible tube directly.

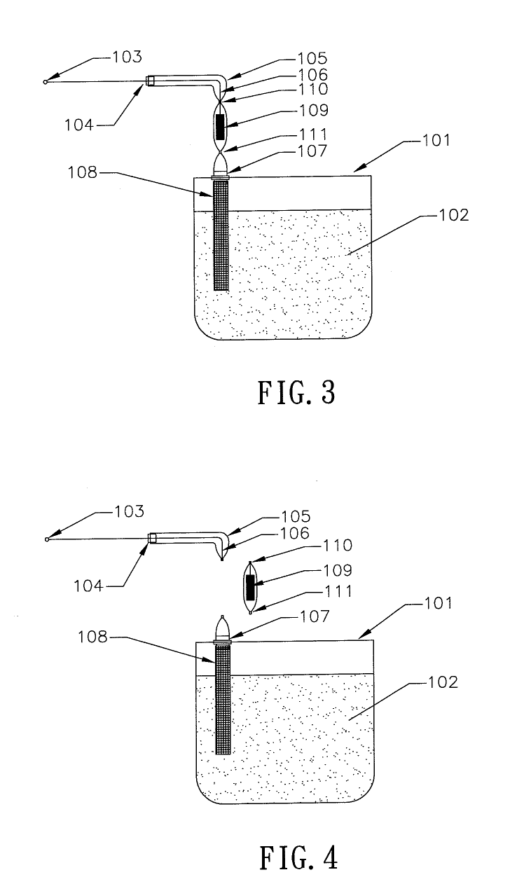

Referring to FIG. 3, a sealer is applied to seal the flexible tube 105 above and under the matrix sample basket 109 and form a sealed zone 110 and 111 to enclose the matrix sample basket 109. In one embodiment, the selection of the sealer or the sealing device for the tube means is as follows. If the packed-bed bioreactor is a single-used packed-bed bioreactor, the tube means can be a thermal settable tube, such as a C-Flex tube, which can be sealed by a heat sealer; and if the packed-bed bioreactor is an autoclavble packed-bed bioreactor, the tube means can be a heat-resisting thermal settable tube, such as a platinum-cured silicon tube or another silicon tube, which can be sealed via compressing the tube means by a clamp.

Referring to FIG. 4, remove the enclosed matrix sample basket 109 by cutting off the sealed zones 110 and 111 above and under the matrix sample basket 109. The matrix sample basket 109 containing embedded cell culture matrixes is then able to send for additional analysis.

Referring to FIG. 5, a detail drawing on the design of the matrix sample basket where 106 is a string that connected on the basket, 109 is the basket with openings, and 109-2 are pre-embedded cell culture matrixes. The dimension of the openings on the basket should be smaller than the dimension of cell culture matrixes 109-2 so that the carriers won't be lost during the collecting process.

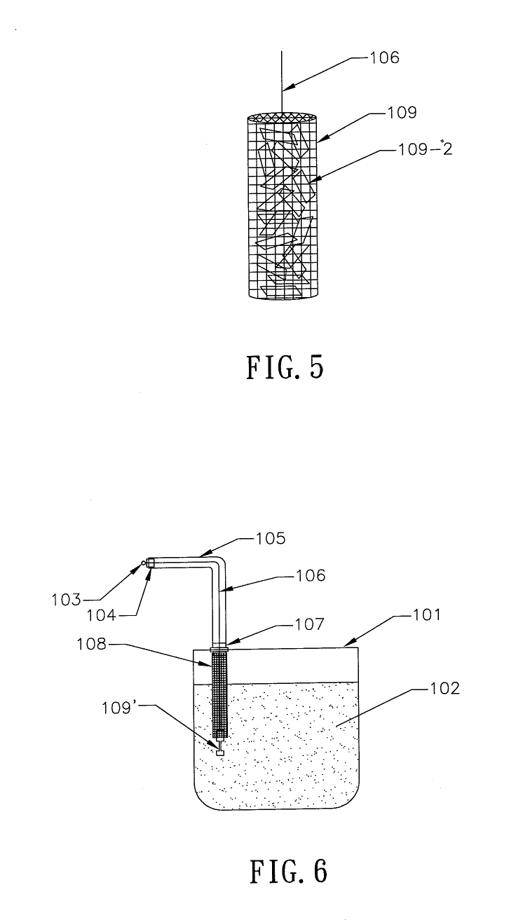

Referring to FIG. 6, the first embodiment of the present invention where the matrix sample basket 109 in FIG. 1 is replaced with a holder 109′ with opening on the side of the holder that is larger than the dimension of the cell culture matrixes in order to collect the cell culture matrixes 102 inside the packed-bed bioreactor.

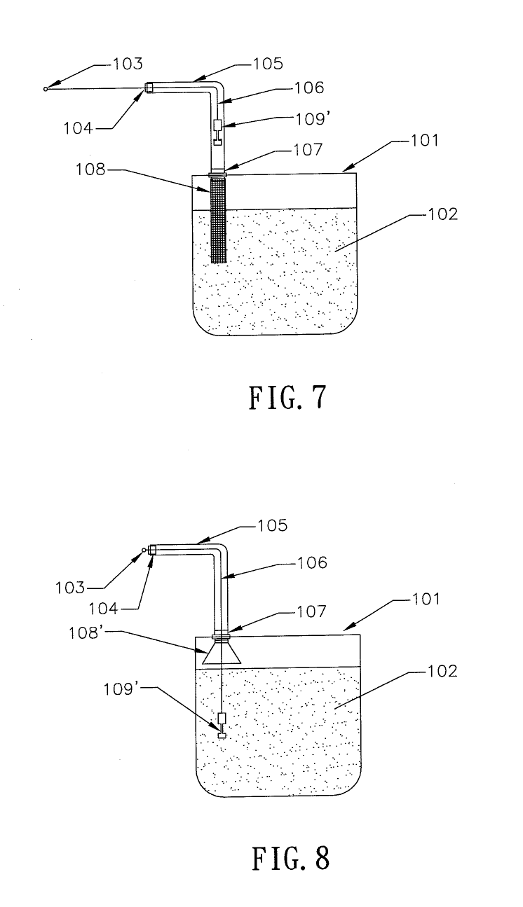

Referring to FIG. 7, the first embodiment of the present invention, when the sampling of cell culture matrix is required, the string 106 is pulled by pulling the knob 103 through the closure 104 and the matrix sample holder 109′ containing the collected cell culture matrixes is pulled into the flexible tube 105 outside the packed-bed bioreactor container 101 through the guiding of the rigid tube 108 inside the container 101.

Referring to FIG. 8, the rigid tube 108 with openings is replaced with a tunnel shape guide 108′ in order to guide the matrix sample basket 109 or matrix holder 109′ from inside the packed-bed bioreactor container 101 to outside the container 101 and into the flexible tube 106.

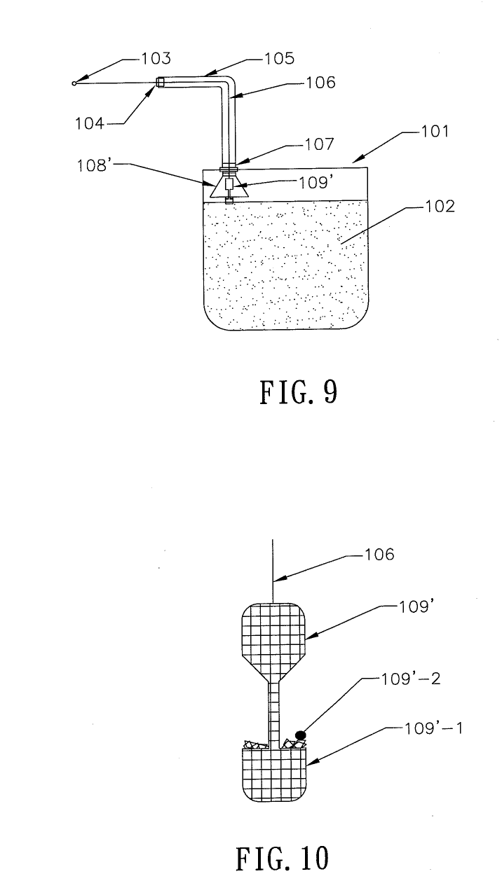

Referring to FIG. 9, the first embodiment of the present invention, when the sampling of cell culture matrix is required, the string 106 is pulled by pulling the knob 103 through the closure 104 and the matrix sample holder 109′ containing the collected cell culture matrixes is pulled into the flexible tube 105 outside the packed-bed bioreactor container 101 by the guiding of the tunnel 108′.

Referring to FIG. 10, the detail drawing of the matrix collecting holder 109′, where it has a holder 109′-1 that could hold the collected cell culture matrixes 109′-2. The holder 109′ is connected with a string 106 that could be pulled up into a flexible tube outside the packed-bed bioreactor.

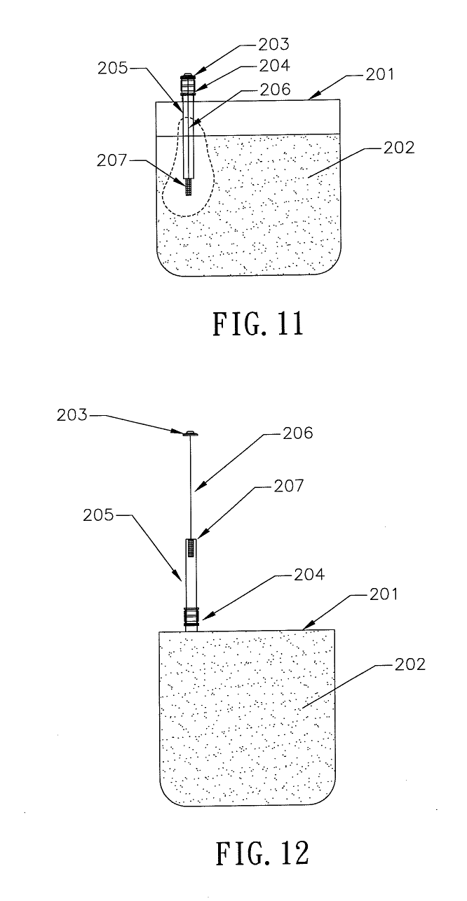

Referring to FIG. 11, the second embodiment of the present invention, a packed-bed bioreactor container 201 packed with cell culture matrixes 202. A single-used matrix sampling device is constructed by a flange cap 203, a flange connector 204, a flexible tube 205 make by thin film of plastic, a string 206 which is fixed between a flange cap 203 and the end of the flexible tube 205. A matrix sample basket 207 containing pre-embedded cell culture matrixes is sealed at the end and outside of the flexible tube 205.

Referring to FIG. 12, when the sampling of cell culture matrixes is required, the flange connector 204 is opened and the flange cap 203 and the string 206 are pulled until the flexible tube 205 is out of the packed-bed bioreactor container 201 and turns inside out where the matrix sample basket 207 is enclosed inside the flexible tube 205.

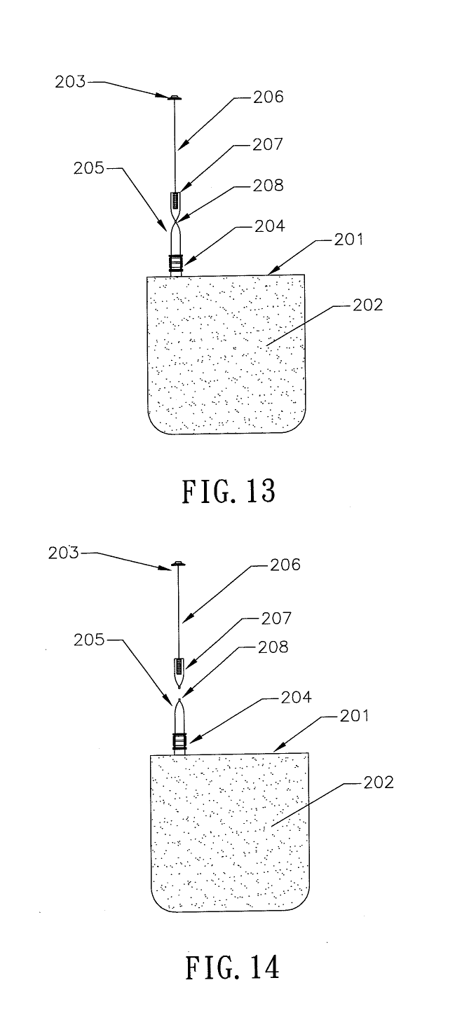

Referring to FIG. 13, a sealer or sealing device is applied to seal the flexible tube 206 under the matrix sample basket 207 and form a sealed zone 208 and make it a closing body containing the matrix sample basket 207. In one embodiment, the selection of the sealer or the seal device for the tube means has been described in above-mentioned embodiment, there is no more explanation necessary herein.

Referring to FIG. 14, remove the enclosed matrix sample basket 207 by cutting off the sealed zones 208 under the matrix sample basket 207. The matrix sample basket 207 containing embedded cell culture matrixes is then able to send for additional analysis.

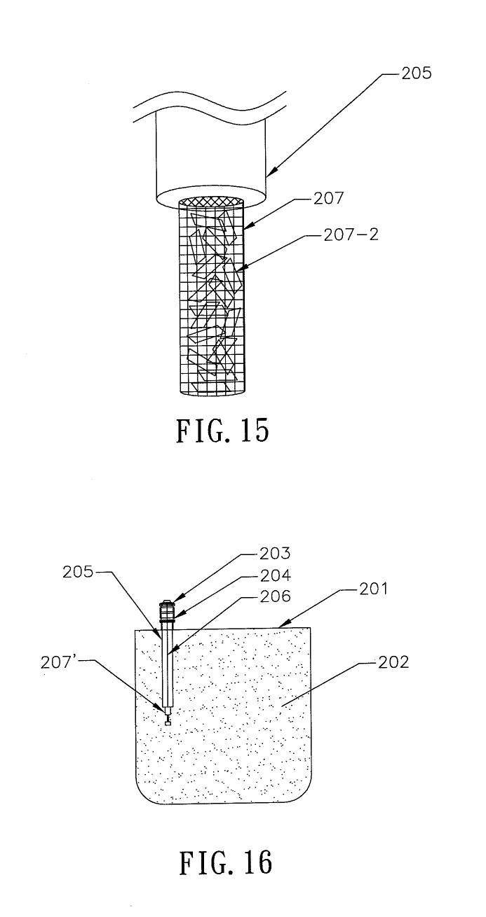

Referring to FIG. 15, the detail design of the matrix sample basket in the second embodiment of the present invention, where 205 is the flexible tube that is made with a thin film of plastic, the matrix sample basket 207 and the cell culture matrixes 207-2 that is pre-embedded inside the basket.

Referring to FIG. 16, the second embodiment of the present invention where the matrix sample basket 207 in FIG. 11 is replaced with a holder 207′ with opening on the side of the holder that is larger than the dimension of the cell culture matrixes in order to collect the cell culture matrixes 202 inside the packed-bed bioreactor container 201.

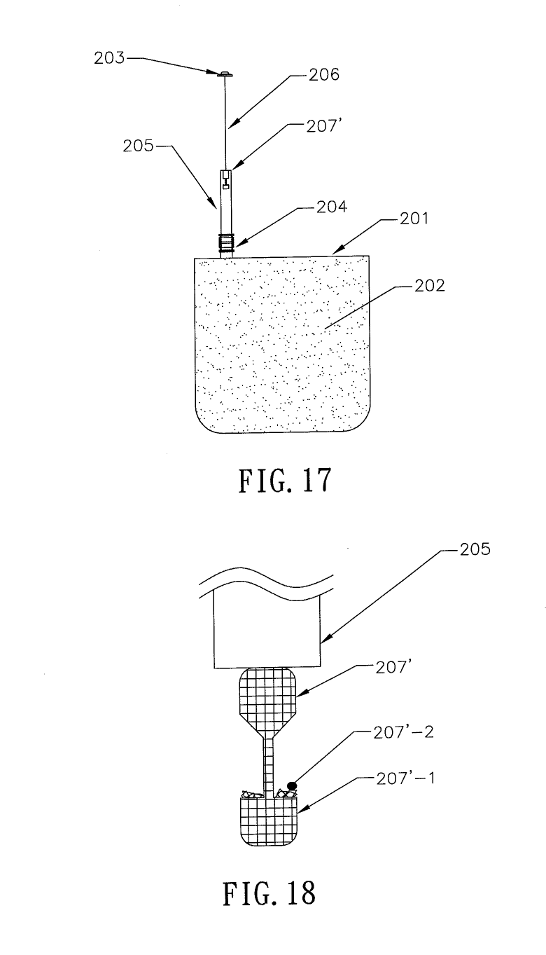

Referring to FIG. 17, the second embodiment of the present invention, when the sampling of cell culture matrix is required, the flange port 204 is opened and pull the flange cap 203 and the string 206 until the flexible tube 205 is out of the packed-bed bioreactor container 201 and turn inside out where the matrix sample holder 207′ is enclosed inside the flexible tube 205.

Referring to FIG. 18, the detail drawing of the matrix collecting holder 207′ of the second embodiment of the present invention, where it has a holder 207′-1 that could hold the collected cell culture matrixes 207′-2. The matrix collecting holder 207′ is connected directly with a flexible tube 205 that could be pulled up and turn inside out to cover the matrix collecting holder 207′ with collected matrixes 207′-2.

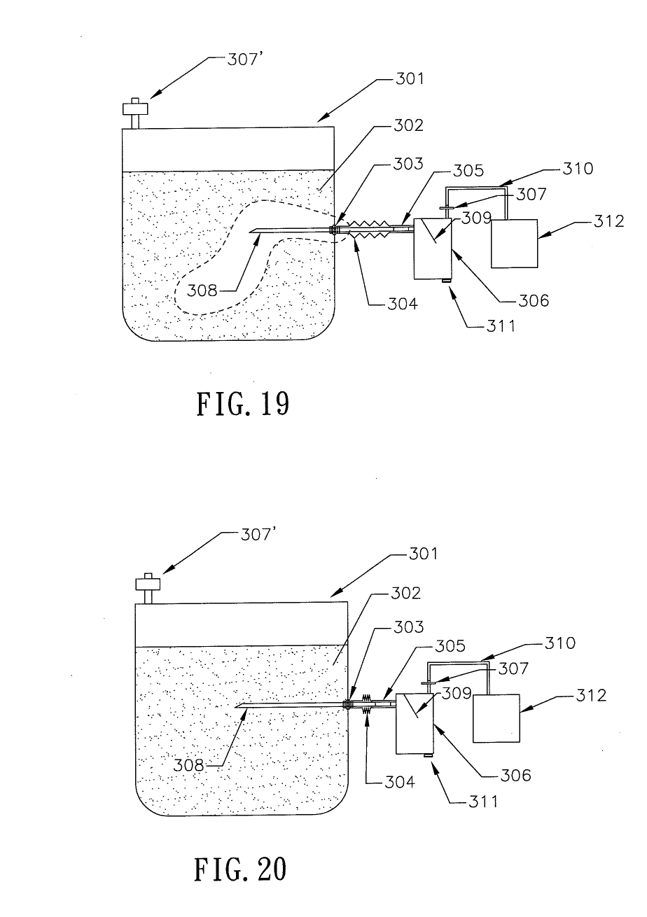

Referring to FIG. 19, the third embodiment of the present invention where 301 is the packed-bed bioreactor container with packed matrixes 302 inside the container 301. An air filter 307′ in the packed-bed bioreactor container 301. An opening 303 mounted on the packed-bed bioreactor container 301 connected to a tube with bellows 304. The tube with bellows 304 is connected to a flexible tube 305 that connect between the sampling rod 308 and the matrix container 306. The matrix container 306 has an air filter 307, an opening with cap 311 at the bottom for releasing the collected matrixes, a hamper 309 to prevent the matrixes to be sucked to block the air filter 307. A vacuum means 312 is connected to the matrix container 306 through a tube 310. The cell culture matrixes 302 inside the packed-bed bioreactor container 301 can be collected through the opening of the sampling rod 308 by generating a vacuum in the matrix container 306.

Referring to FIG. 20, the third embodiment of the present invention where the sampling rod 308 can move to and fro in the packed-bed bioreactor 301 to collect the matrixes 302 in different region inside the packed-bed bioreactor 301 by pushing and pulling the tube with bellows 304 surrounding the sampling rod 308.

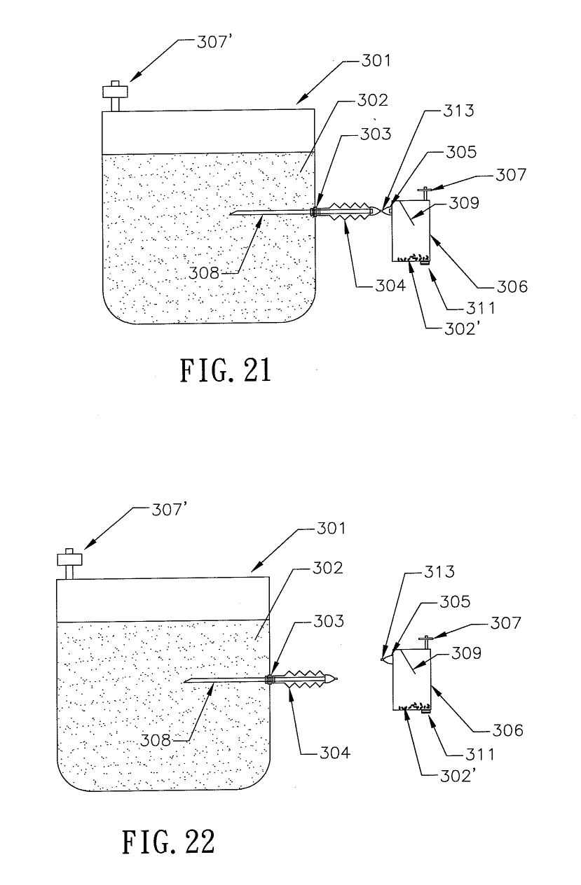

Referring to FIG. 21, the third embodiment of the present invention where after the matrixes 302′ are collected into the matrix container 306 by the suction force with vacuum, the flexible tube between the sampling rod 308, the tube with bellows 304 and the matrix container 306 is sealed through a sealing means. A sealing zone 313 is form after the flexible tube is sealed. In one embodiment, the selection of the sealer or the seal device for the tube means has been described in above-mentioned embodiment, there is no more explanation necessary herein.

Referring to FIG. 22, the third embodiment of the present invention where matrix container 306 is removed by cutting off the sealed zone 313 with a cutting means. The matrix container 306 containing collected cell culture matrixes 302′ is then able to send for additional analysis. The collected matrix 302′ can be released from the matrix container 306 through the opening 311 in a biosafety cabinet.

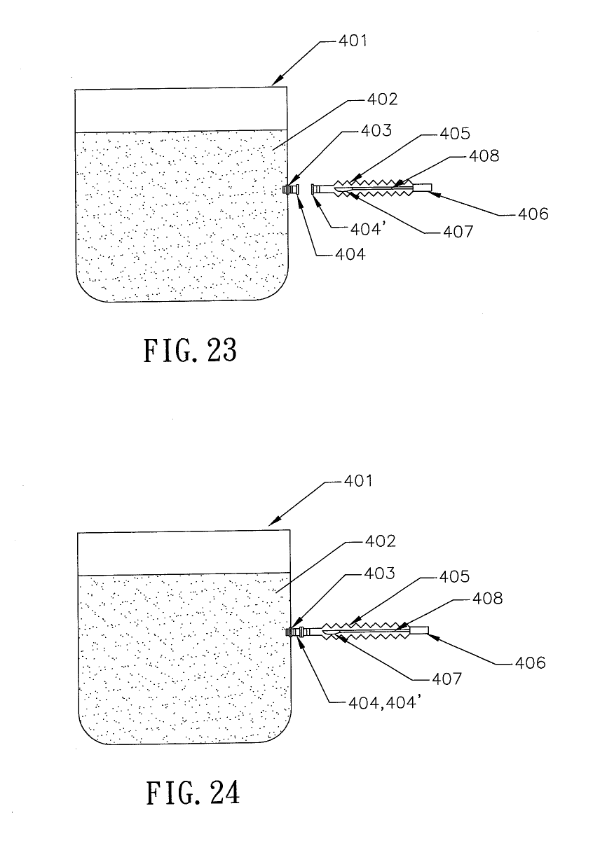

Referring to FIG. 23, the fourth embodiment of the present invention, a packed-bed bioreactor container 401 packed with cell culture matrixes 402. An opening 403 connected with a sterile connector 404 is mounted on side or top of the container 401. A single-used matrix sampling device is constructed by a tube with bellows 405, a scoop 407 inside the tube with bellows 405, a rod 408 connected between the scoop 407 and a handle 406. A sterile connector 404 is connected at the other end of the tube with bellows 405. The sterile connector can be any sterile connectors from ReadyMat or Kleenpak or the like.

Referring to FIG. 24, the fourth embodiment of the present invention, where the single-used matrix sampling device is connected with the packed-bed bioreactor container 401 through the sterile connector 404, 404′.

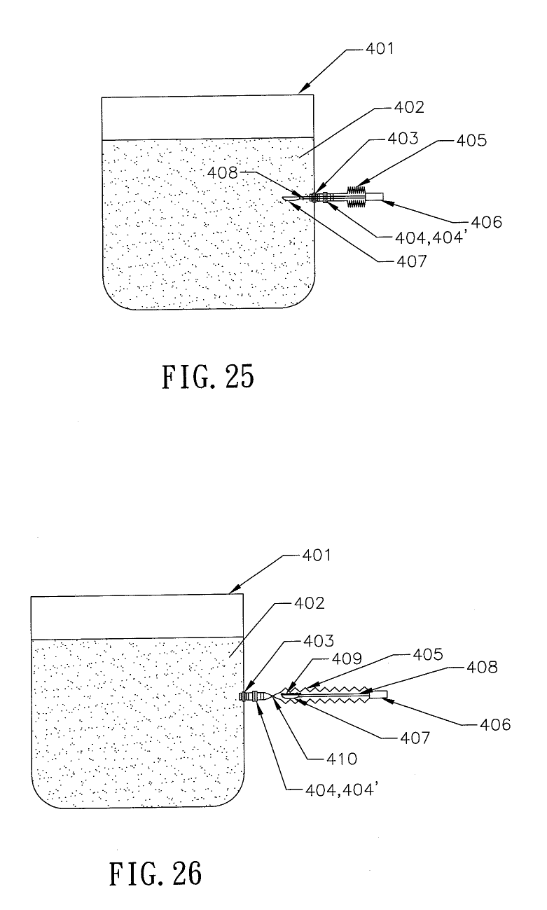

Referring to FIG. 25, the fourth embodiment of the present invention after compressing the tube with bellows 405 and allow the scoop 407 to enter into the packed-bed bioreactor container 401 to collect the matrixes 402.

Referring to FIG. 26, after collecting the cell culture matrix 402 from the packed-bed bioreactor container 401 with the scoop 407, the tube with bellows 405 is extended and the scoop 407 containing collected cell culture matrixes 409 is moved back to the tube with bellows 405 and surround by the tube with bellows 405. The tube with bellows between the packed-bed bioreactor tank opening 403 with sterile connectors 404, 404′ and the scoop 407 is sealed through a sealing means. A sealing zone 410 is form after the flexible tube with bellows is sealed. In one embodiment, the selection of the sealer or the seal device for the tube means has been described in above-mentioned embodiment, there is no more explanation necessary herein.

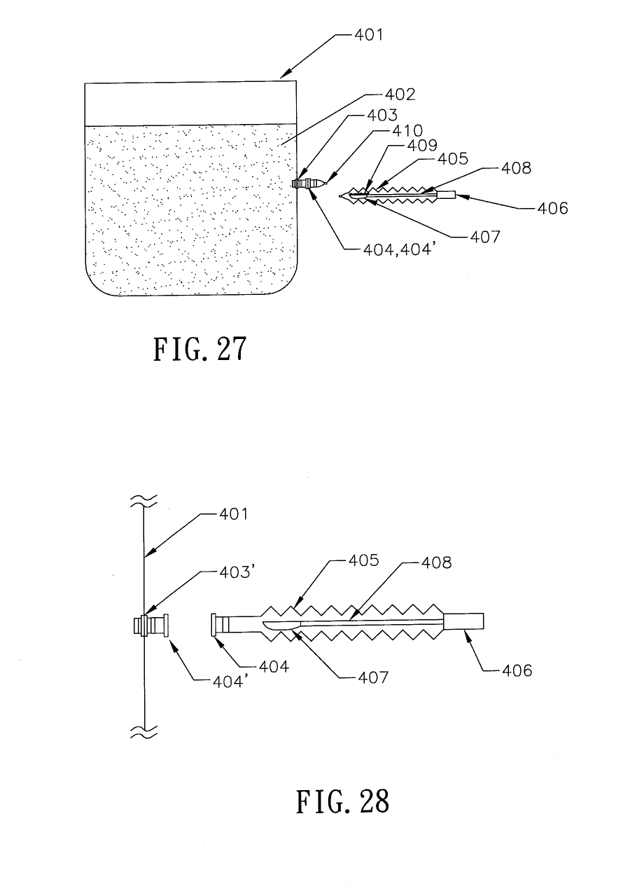

Referring to FIG. 27, the fourth embodiment of the present invention where the single-used matrix sampling device is removed by cutting off the sealed zone 410 with a cutting means. The single-used matrix sampling device containing collected cell culture matrixes 409 is then able to send for additional analysis.

Referring to FIG. 28, the detail drawing of the matrix collecting device. The matrix collecting device is constructed by a flexible tube with bellows 405, a sterile connector 404 such as ReadyMat or Kleenpak sterile connector on the one end of the flexible tube with bellows 405, a scoop 407 connected by a rigid rod 408 that is placed inside the flexible tube with bellows 405. A handle at the other end of the flexible tube with bellows 405 which connects the rigid rod 408 with scoop 407 is sealed by the flexible tube with bellows 405. In order for the matrix collecting device to connect with the packed-bed bioreactor in order for matrix sample, a panel mount connector 403′ with two-end opening that is mounted on the packed-bed bioreactor wall 401. A sterile connector 404′ such as ReadyMat or Kleenpak sterile connector is connected to the panel mounted connector 403′ with two-end openings.

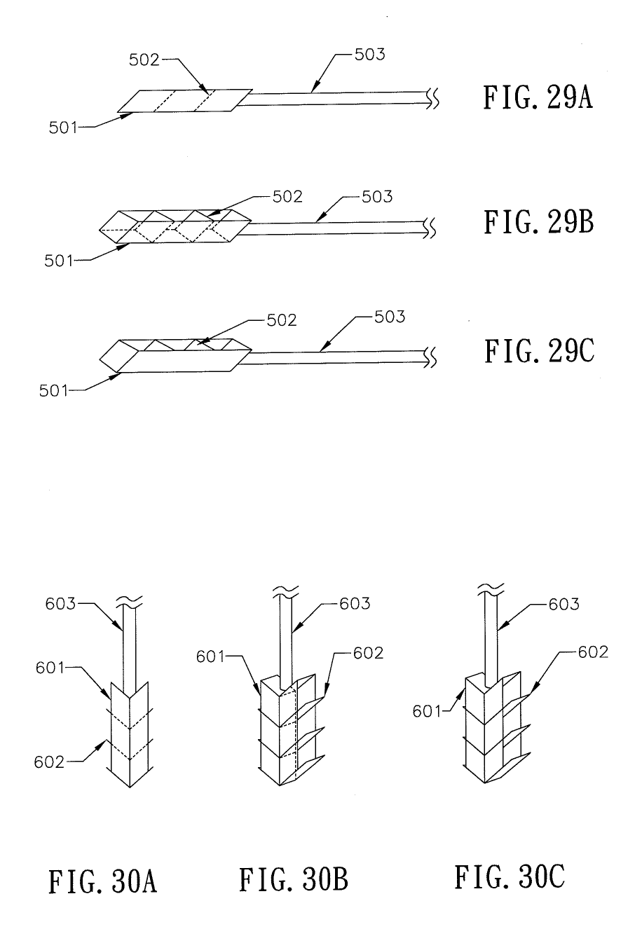

Referring to FIGS. 29A, 29B and 29C, more options on the design of the sampling device. For example, if the sampling device is designed to take sample in horizontal direction, a scoop in a parallelogram shape from side view with a holder 501 and multiple slanting boards 502 inside the holder 501 is designed. The parallelogram shape of the holder 501 makes the sampling device easier to move forward into the packed-bed with cell culture matrixes; where the slanting boards are able to stop the matrixes to get out when moving the sampling device outward.

Referring to FIGS. 30A, 30B, and 30C, more options on the design of the sampling device. For example, if the sampling device is designed to take sample in vertical direction, a scoop or holder with vessel 601 and multiple openings on two sides is designed. The openings are formed by placing multiple slanting boards 602 inside the vessel 601. The slanting boards 602 are extruded out of the vessel 601 so that it will be easier to guide the cell culture matrixes into the vessel 601 for collection. Another example of sampling device can be a forceps or a loop connected and controlled by a wire through the center of the hollow rigid/flexible tube to another end of wire externally. The device can reach anywhere in the matrix bed to collect the sample. As the samples are secured by the device, they can be aseptically removed as stated previously for further study.

In summary, the single-used matrix sampling device and method according to the present invention provides a tube means and a matrix collecting means for sampling the cell culture matrixes aseptically from the packed-bed bioreactor, especially for the purpose of single-used, disposable or autoclavble packed-bed bioreactor, in order to do cell analysis including cell count, viability, contamination, cell characterization, . . . etc. without requiring to face the threaten of contamination.

While the invention is susceptible to various modifications and alternative forms, a specific example thereof has been shown in the drawings and is herein described in detail. It should be understood, however, that the invention is not to be limited to the particular form disclosed, but to the contrary, the invention is to cover all modifications, equivalents, and alternatives falling within the spirit and scope of the appended claims.

What is claimed is:

1. A single-used matrix sampling device for a packed-bed bioreactor which comprises a closed container to receive a cell culture matrix, said single-used matrix sampling device comprising:
a matrix collecting means for sampling said cell culture matrix, said matrix collecting means comprising a receptacle and a pulling means configured to said receptacle at one end thereof for moving said receptacle outside said container; and
a tube means for preventing said matrix collecting means from being contaminated while sampling, and said tube means configured to said container at one end with a connector, wherein said tube means is sealed to enclose said receptacle after said receptacle is pulled outside said container through said connector to said tube means.

2. The single-used matrix sampling device according to claim 1, wherein said packed-bed bioreactor is a single-used packed-bed bioreactor, said tube means is thermal settable tube which can be sealed by a heat sealer.

3. The single-used matrix sampling device according to claim 1, wherein said packed-bed bioreactor is an autoclavble packed-bed bioreactor, said tube means can be a heat-resisting thermal settable tube which can be sealed via compressing by a clamp.

4. The single-used matrix sampling device according to claim 1, wherein said receptacle comprises a holder with at least an opening and a basket which contains a pre-embedded cell culture matrix.

5. The single-used matrix sampling device according to claim 1, wherein said tube means is mounted outside said container.

6. The single-used matrix sampling device according to claim 5, wherein a second end of said tube means has a robber stopper with a resilient opening that said pulling means is passed for pulling said receptacle by an external force.

7. The single-used matrix sampling device according to claim 5, wherein said tube means is made by plastic film and a second end of said tube means is closed so that said pulling means is pulled inside said tube means.

8. The single-used matrix sampling device according to claim 5, wherein said tube means further comprises a bellows for changing the length of said tube means.

9. The single-used matrix sampling device according to claim 8, wherein said matrix collecting means is set inside said tube means with one end fixation and a second end of said tube means has a sterile connector for connecting with said connector; and said matrix collecting means is inserting into or pulling out said container through said sterile connector and said connector.

10. The single-used matrix sampling device according to claim 9, wherein said matrix collecting means is in a scoop-like shape.

11. The single-used matrix sampling device according to claim 1, wherein
said tube means is made by thin film of plastic to function as a flexible tube and is mounted inside said container with said connector which functions as a flange connector;
the other end of said pulling means is fixed between a flange cap and the other end of said flexible tube and is disposed inside said flexible tube, and said receptacle is set on an external surface of said flexible tube at said other end thereof for collecting said cell culture matrix; and
said flexible tube is sealed to enclose said receptacle after said receptacle and said flexible tube are pulled outside said container through said flange connector and said flexible tube are turned inside out so that said receptacle is covered by said external surface of said flexible tube.

12. A single-used matrix sampling device for a packed-bed bioreactor which comprises a container which is closed to receive a cell culture matrix, said single-used matrix sampling device comprising:
a matrix collecting means for sampling said cell culture matrix, wherein said matrix collecting means comprises a matrix container equipped with a vacuum means and a sampling rod, wherein said sampling rod is extended into said container surrounded with said cell culture matrix through an opening on said container to guide said cell culture matrix sucked into said matrix container; and
a tube means for preventing said matrix collecting means from being contaminated while sampling, wherein said tube means is connected between said sampling rod and said matrix container; and said tube means is sealed to enclose said matrix container after said cell culture matrix is sucked out said container through said connector to said matrix container by said vacuum means. (FIG. 19-22)

13. The single-used matrix sampling device according to claim 12, wherein said tube means further comprises a bellows portion connected to said tube means to cover a portion of said sampling rod outside said container, and said bellows portion can be extended or shorten so that said sampling rod can be moved around said container.

14. The single-used matrix sampling device according to claim 1, wherein said single-used matrix sampling device is in a parallelogram shape from side view, and said single-used matrix sampling device comprises a holder and a plurality of slanting boards inside said holder.

15. The single-used matrix sampling device according to claim 1, wherein said single-used matrix sampling device comprises a scoop or holder with a vessel and a plurality of openings on two sides; said openings are formed by placing a plurality of slanting boards inside said vessel; and said slanting boards are extruded out of said vessel.

16. A single-used matrix sampling method, comprising the following step:
providing a single-used matrix sampling device for a packed-bed bioreactor which comprises a closed container to receive a cell culture matrix, said single-used matrix sampling device comprising:
a matrix collecting means for sampling said cell culture matrix, said matrix collecting means comprising a receptacle and a pulling means configured to said receptacle at one end thereof for moving said receptacle outside said container; and
a tube means for preventing said matrix collecting means from being contaminated while sampling, and said tube means configured to said container at one end with a connector, wherein said tube means is sealed to enclose said receptacle after said receptacle is pulled outside said container through said connector to said tube means.;
putting said matrix collecting means into said container to submerge said receptacle in said cell culture matrix;
pulling out said receptacle containing said cell culture matrix from said container into said tube means by said pulling means;
sealing said tube means to enclose said receptacle to form a enclosed matrix sample by a heat sealing method or a compressing method and cutting off the sealed zone of said tube means to separate sealed said tube means from said packed-bed bioreactor; and
taking out said enclosed matrix sample from the sealed tube means for further analysis.

17. The single-used matrix sampling method according to claim 16, wherein the other end of said tube means has a robber stopper with a resilient opening that said pulling means is passed through; and said receptacle is pulled outside said container by pulling said pulling means through said resilient opening.

18. The single-used matrix sampling method according to claim 16, wherein said tube means is made by plastic film and the other end of said tube means is closed so that said pulling means is pulled inside said tube means.

19. The single-used matrix sampling method according to claim 16, wherein said tube means further comprises a bellows portion for changing the length of said tube means; said matrix collecting means in a scoop-like shape is set inside said tube means with one end fixation and the other end of said tube means has a sterile connector for connecting with said connector; when said sterile connector is connected to said connector; and said step for putting said matrix collecting means into said container to submerge said receptacle in said cell culture matrix comprises the step for compressing said bellows portion to insert said matrix collecting means into said container via said sterile connector and said connector.

20. A single-used matrix sampling method comprising the following step:
providing a single-used matrix sampling device for a packed-bed bioreactor which comprises a container which is closed to receive a cell culture matrix, said single-used matrix sampling device comprising:
a matrix collecting means for sampling said cell culture matrix, wherein said matrix collecting means comprises a matrix container equipped with a vacuum means and a sampling rod, wherein said sampling rod is extended into said container surrounded with said cell culture matrix through an opening on said container to guide said cell culture matrix sucked into said matrix container; and
a tube means for preventing said matrix collecting means from being contaminated while sampling, wherein said tube means is connected between said sampling rod and said matrix container; and said tube means is sealed to enclose said matrix container after said cell culture matrix is sucked out said container through said connector to said matrix container by said vacuum means;
putting said matrix collecting means into said container to submerge said sampling rod in said cell culture matrix;
vacuuming out said cell culture matrix from said container through said connector to said matrix container by said vacuum means;
sealing said tube means to enclose said matrix container to form a enclosed matrix sample by a heat sealing method or a compressing method and cutting off the sealed zone of said tube means to separate sealed said tube means from said packed-bed bioreactor; and
removing said vacuum means and taking out said enclosed matrix sample from the sealed tube means for further analysis.

21. The single-used matrix sampling method according to claim 20, wherein said tube means further comprises a bellows portion connected to said tube means to cover a portion of said sampling rod outside said container; and said step for putting said matrix collecting means into said container to submerge said receptacle in said cell culture matrix comprises compressing said bellows portion so that said matrix collecting means can be moved around said container.
